(19) United States
(12) Patent Application Publication
(10) Pub. No.: US 2012/0232364 A1
(43) Pub. Date: Sep. 13, 2012

(54) METHOD AND APPARATUS FOR OPTOACOUSTIC MONITORING OF BLOOD COMPONENTS IN A BLOOD FLOW

(75) Inventor: J. Michael Delmage, Napa, CA (US)

(73) Assignee: TRANSVIVO INC., Richmond, CA (US)

(21) Appl. No.: 13/413,879

(22) Filed: Mar. 7, 2012

Related U.S. Application Data
(60) Provisional application No. 61/450,302, filed on Mar. 8, 2011.

(51) Int. Cl. A61B 5/1455

(52) U.S. Cl. 600/311; 600/322; 600/316; 600/317

(57) ABSTRACT

Described is an apparatus and method of optoacoustic monitoring of blood concentrations of one or more constituents by directing a flow of a patient's blood through a substantially transparent vessel to optoacoustically detect a concentration of one or more constituents. To detect constituents, pulses of laser light can be passed through the blood flow at one or more frequencies in order to generate an altered laser emission from the exposed blood, and/or induce detectable optoacoustic responses from the constituents. The detectable responses can be detected and measured by analyzing an alteration of the laser emissions and/or the frequency, slope and/or amplitude of the optoacoustic responses for different constituents in the blood.

### CROSS-REFERENCE TO RELATED APPLICATIONS

This application claims priority to U.S. Provisional Application No. 61/450,302 filed Mar. 8, 2011 and incorporated herein by reference.

### BACKGROUND OF THE INVENTION

The state of a person's health may be indicated by the constituents and constituent concentrations of the person's blood. For example, the concentration of various electrolytes including potassium, phosphates, calcium, urea, glucose, etc. are important factors in determining the present state of health as well as indicating the potential for future health problems, their diagnosis and prevention, possibly well before symptoms occur. It may also be desirable to continuously monitor presence and/or concentrations of harmful substances such as drugs, poisons or disease-related components, for example, pathogens, toxins, bacteria, viruses and antibodies, as part of a patient treatment regimen. Although blood sampling and testing are frequently carried out for such purposes, the testing is not continuous but instead is static with the results indicating the constituent concentrations of a single blood draw sample at a time. Moreover, such sampling is not typically repeated frequently during ongoing treatments or during therapy at frequent intervals to determine the real-time or continuous effectiveness of the treatment or therapy.

### SUMMARY OF THE INVENTION

The methods and apparatus of the present invention provide a continuous, real-time monitoring and evaluation of a patient's present and/or progressing health, improvement, or deterioration by monitoring a patient's blood flow for a selected predetermined period of time. Specific constituents may be identified, selected and monitored such as electrolytes as well as albumin, proteins, poisons, drugs, disease-related components or other identifiable constituents. The method comprises optoacoustic monitoring of a flow of a patient's blood through a substantially transparent tube, passing pulses of laser light of one or more selected wavelengths through the transparent tube containing the blood flow to induce ultrasonic wave responses from the selected constituents, and detecting and measuring the ultrasonic optoacoustic responses induced from the one or more constituents in response to the laser beam, and/or measuring laser beam amplitude and/or wavelength alteration created by the one or more constituents in response to a laser beam(s).

An optoacoustic response is induced when a constituent of the effluent absorbs lights from a laser pulse of a certain wavelength, is heated and thermally expands, creating an ultrasonic, photoacoustic (optoacoustic) wave. The wave is monitored by ultrasonic detectors as will be described in more detail hereinafter. The induced optoacoustic response is also measured and the concentration of the constituent(s) determined or calculated from the measured response.

In one embodiment, the pulses of laser light are directed through the transparent tube through which the patient's blood flows at one or more frequencies for inducing detectable optoacoustic waves for one or more of the constituents, respectively, and the frequency, slope and/or amplitude of optoacoustic waves generated by the constituents are detected and measured by ultrasound detectors.

In another embodiment, the method comprises determining laser beam wavelengths that induce optoacoustic detectable responses from selected different constituents, respectively, based on a respective constituent concentration, and determining the concentration of a blood constituent based on the measured optoacoustic wave frequency, slope and/or amplitude.

In one embodiment, the method comprises detecting and measuring laser beam amplitude change and/or laser beam wavelength alteration or shift for one or more different selected laser beams passing through the constituent containing blood flow, and correlating amplitude change and/or wavelength alteration with corresponding optoacoustic responses and/or constituent concentrations, respectively.

In one embodiment, a dye, ligand or marker configured to cooperate with one or more of blood constituents is introduced into a blood flow for enhancing optoacoustic responses to laser light pulses.

In another embodiment, a method comprises determining optimum laser beam wavelengths for generating optoacoustic responses for one or more of the blood constituents, respectively, and directing the optimum laser beam wavelengths into the blood flow containing tube and detecting and measuring the optoacoustic wave frequency, slope and/or amplitude generated by the one or more constituents in response to the respective optimum laser beam wavelengths.

In one embodiment, the method comprises determining optimum laser beam wavelengths for generating altered laser emissions for one or more of the constituents, respectively, and directing the optimum laser beam wavelength into the blood flow containing tube and detecting and measuring the altered laser emissions generated in response to the optimum laser beam wavelength(s).

In another embodiment, the method comprises injecting a detectable biologic marker into the patient's blood upstream of the transparent tube, the marker configured to cooperate with one or more of the constituents for enhancing optoacoustic responses to the laser light pulses.

In other embodiments, apparatus configured to generate optoacoustic responses for measuring the concentrations of the one or more constituents in a bloodstream utilizing the aforesaid methods are described. Such apparatus includes diagnostic circuits incorporating an optoacoustic monitor, as well as dialysis or therapeutic apheresis systems.

### BRIEF DESCRIPTION OF THE DRAWINGS

FIG. 1 is a schematic illustration of a basic blood flow monitoring circuit including an optoacoustic monitoring apparatus;

FIG. 2 is an enlarged schematic side view illustration of an optoacoustic sensor module of an optoacoustic monitoring apparatus of FIG. 1 showing laser light and pressure transducer components;

FIG. 3 is a front view of the optoacoustic sensor module of FIG. 2;

FIG. 4 is a schematic illustration of a typical chronic dialysis machine with an external stand-alone optoacoustic monitoring apparatus secured adjacent to the machine;

FIG. 5 is a schematic illustration of a dialysis machine with an integrated optoacoustic monitoring apparatus;

FIG. 6 illustrates an optoacoustic sensor module of FIG. 2 mounted on blood flow tubing in a hemofiltration apparatus upstream of a hemofilter;

FIG. 7A is a schematic view of a portion of therapeutic apheresis apparatus illustrating use of optoacoustic monitor components;

FIG. 7B is a schematic view of a portion of apparatus for patient fluid management incorporating optoacoustic monitor components; and

FIG. 8 is a schematic illustration of CRRT apparatus illustrating another configuration of locating optoacoustic monitoring components.

### DETAILED DESCRIPTION OF EMBODIMENTS

In the following description of apparatus, the same components or devices may be referred to by the same reference numeral in the different drawings.

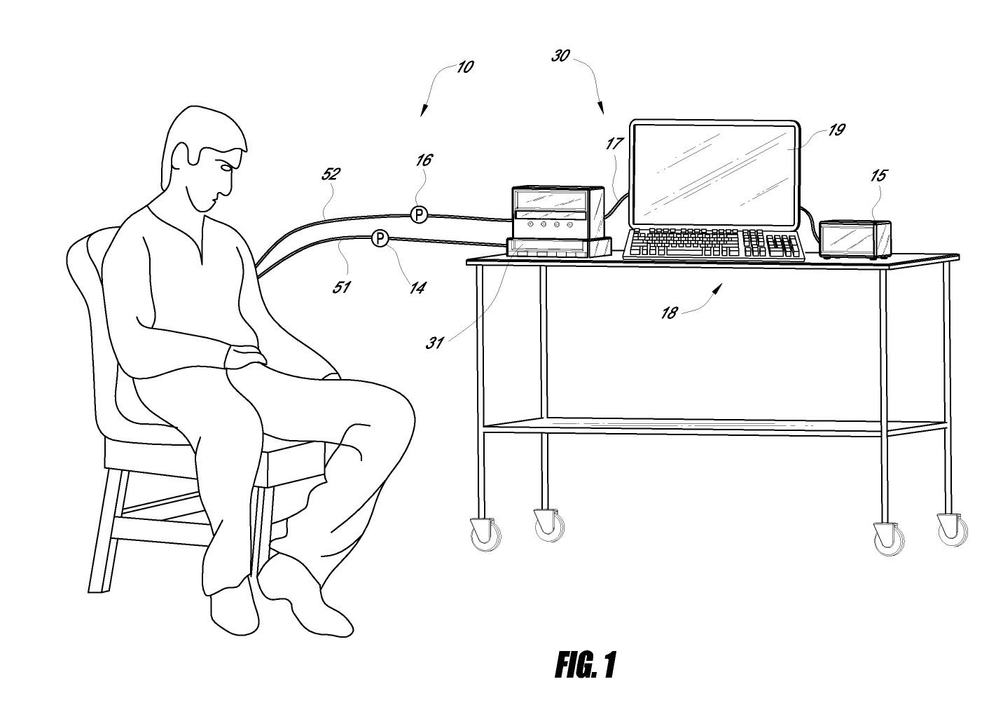

In FIG. 1, there is illustrated a blood flow monitoring circuit 10 including blood access tubing 51 and blood return tubing 52 configured for directing blood withdrawn from a patient's vein to a testing apparatus. An optoacoustic monitoring apparatus 30 is positioned along the circuit tubing for monitoring constituents in the blood flow as will be explained further. Blood withdrawn from a patient is directed along circuit tubing 17 using blood pumps 14 and 16. The blood access and return lines, 51, 52, respectively, are provided with appropriate components such as needles communicating with suitable peripheral veins, for example, needles typically used for blood sampling or IV therapy. Pumps 14 and 16 may be peristaltic pumps such as used for dialysis machines. However, any means for withdrawing the blood from a patient, including tubing and suitable pumps for directing the withdrawn blood to the optoacoustic monitoring components and returning the blood to the patient thereafter in a substantially continuous blood flow, may be used.

The FIG. 1 schematic optoacoustic monitoring apparatus 30 configuration comprises a computer 18 cooperating with the optoacoustic sensor 31 including a monitor 19 for displaying results including measurements and concentrations of detected and monitored blood constituents. A suitable computer may also include microprocessors or similar components including software programs configured for calculating constituent concentrations based on optoacoustic measurements of optoacoustic signals including wave frequency, slope and/or amplitude and/or laser beam wavelength alteration or shift detected by the optoacoustic apparatus. A suitable computer may also be provided with software or programs for comparing and correlating the amplitude and/or wavelength shifts with acoustic wave amplitudes. Thus, the optoacoustic monitoring system may be capable of detecting the presence of one or more blood constituents as well as calculating and displaying concentrations of the constituents as the blood flows through the optoacoustic monitoring components. Such monitoring may be accomplished continuously as the blood flows through the system, or intermittently at selected intervals. The apparatus may also be configured to operate the pumps for withdrawing and returning the blood continuously, or at selected intervals.

In FIG. 1, there also is schematically illustrated an embodiment for transmission of processed data signals from the optoacoustic monitor apparatus to a remote location. In the embodiment illustrated, a modem 15 is connected to computer 18 which is connected and communicates with the optoacoustic sensor 31. The modem provides means for transmission of the processed data signals received from the computer through telephone lines or otherwise transmitted to a modem, display and/or printing equipment at a remote location. Alternatively, wireless transmission may be used, for example where the transmission is through computer connections and/or router to a central processing and/or other monitoring location. Remote transmission of the optoacoustic monitored and processed signals is valuable for giving a physician, technician or other operator real-time and continuing monitoring of the dialysis efficiency as well as detecting and determining adjustments needed to correct and/or maintain patient blood constituents within desired concentration limits and acceptable levels. Such information may be useful in determining and prescribing different treatment therapy as well as modification of dialysis procedures and/or schedules. Such transmission to a remote location may also be used with apparatus embodiments and configurations described hereinafter.

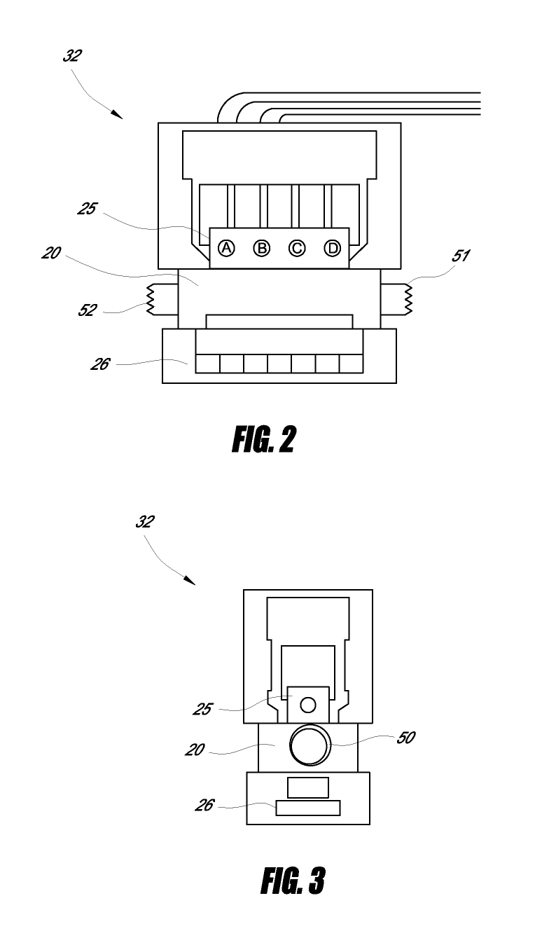

Referring also to FIGS. 2 and 3, the optoacoustic sensor 31 shown in FIG. 1, comprises an optoacoustic sensor module 32 including a substantially transparent tube 20 cooperating with a bank of lasers 25 configured for directing pulses of laser light through the transparent tube and blood flowing within the tube. The material for the substantially transparent tube 20 may be any suitable material which will not distort or impede laser light beams, for example, glass, transparent plastic, or other substantially transparent materials through which a blood supply may continuously flow. The term “tube” as used herein is intended to include any vessel or conduit through which blood may be directed. The tube need not be cylindrical but of any suitable shape and size. The tube is also preferably provided with necessary adaptors or fittings for securing the blood supply tubing to and from the sensor module.

Four lasers, A, B, C and D are shown in the FIG. 2 configuration. However, other numbers of lasers may be used to meet the apparatus requirements. Examples of lasers which may be suitable include Nd:YAG lasers, Ti:Sapphire, Alexandrite laser, Ruby laser, capable of generating short optical pulses. Another example of a suitable pulsed light source is a compact optical parametric oscillator system which generates pulsed tunable Infrared radiation in wavelength ranges between about 600 nm and about 2440 nm, a pulse duration about 10 ns, and a repetition rate about 20 Hz. An example of a commercially available system is Opolette 532 II (Opotek Inc., Carlsbad, Calif.). Such equipment is further described in U.S. Pat. No. 6,295,160, the description of which is incorporated herein by reference.

Detection of generated ultrasonic waves generated by blood components in response to the laser light beams may be performed by an ultrasound detector comprising piezoceramic or piezoelectric pressure transducers or optical detectors 26 schematically illustrated in FIGS. 2 and 3. An array of such detectors shown is configured opposite the laser light sources, although other configurations may be used. The optoacoustic signals may be amplified and digitized or otherwise generated and modified to be used to calculate and display the desired constituent concentrations. Examples of lasers and an optoacoustic sensing apparatus are described in U.S. Patent Application Publication No. 2008/0255433, the description of which is incorporated herein by reference. Another known optoacoustic apparatus is described in Proc. of SPIE, Vol. 7564, 7564 1H, “Noninvasive Optoacoustic Monitoring Platform,” Esenaliev, et al. © 2010.

In the apparatus illustrated in the schematic drawings of FIGS. 1-3, the number and array of lasers for providing pulsed laser beams, as well as the array of pressure transducers, is shown by illustration only. For example, the four different laser lights may each produce a single different wavelength of light selected to create a desired optoacoustic response. Alternatively, different lasers which may be tuned may be advantageous for scanning the blood flowing through the transparent tube, thereby highlighting the different selected constituents to be monitored.

Optical detection techniques and apparatus for ultrasound detection may be used in addition to or as an alternative to the piezoceramic or piezoelectric transducer detectors. Examples of such optical ultrasound detectors comprise an optical etalon or Fabry-Perot etalon or interferometer. Another example of an optical sound sensor is a high-bandwidth optical ultrasound sensor described in J. Biomed Opt. 2011, January-February; 16(1); and Photoacoustic Imaging and Spectroscopy, CRC Press, © 2009 by Taylor & Francis Group LLC. Fabry-Perot etalons are commercially available from LightMachinery, Nepean, Ontario, Canada and more information can be found on the Internet at lightmachinery.com.

In one embodiment, the optoacoustic monitoring apparatus also includes means for amplifying the detected ultrasonic waves, and, preferably, digitizing the signals. A specific example of a useful amplifier is a low-noise 20-dB preamplifier (Onda Corp., Sunnyvale, Calif.) and a low-noise 40-dB amplifier (Analog Modules Inc., Longwood, Fla.). An example of a useful digitizer is a 100-MHz 8-bit digitizer (NI-5112, National Instruments Corp., Austin, Tex.).

Referring again to the embodiment shown in FIG. 1, the optoacoustic monitoring apparatus computer 18 may include the aforesaid amplifier and digitizer to which the signals are sent from the optoacoustic sensor 31. Alternatively, the amplifier and/or digitizer may be separate components. As previously described, a suitable computer comprises microprocessor or components provided with software or programs configured for processing the digitalized signals and calculating constituent concentrations based on optoacoustic signals including wave frequency, slope and/or amplitude detected by the sensor 31.

As previously described, the apparatus for monitoring concentrations of one or more blood constituents in a continuous stream of blood includes a blood monitoring configuration like that schematically illustrated in FIG. 1 or may further comprise other blood flow directing equipment, for example, a dialysis machine for carrying out continuous renal replacement therapy (CRRT). In such a circuit, the optoacoustic apparatus may be installed along the blood supply tubing for periodic or continuous monitoring of blood constituents during the dialysis treatment.

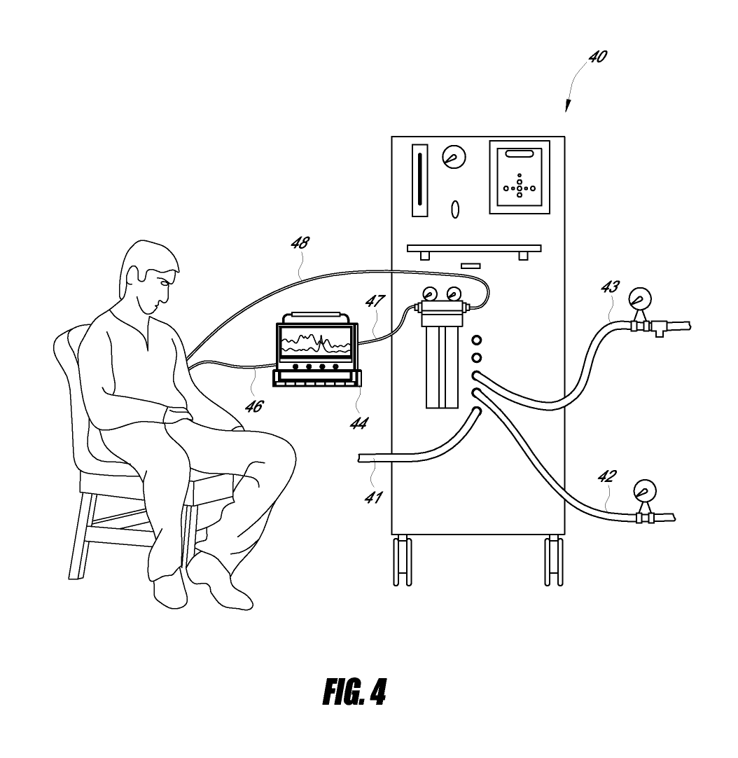

FIG. 4 illustrates the use of a stand-alone optoacoustic monitoring apparatus used with a typical chronic dialysis machine. In the configuration shown, the patient is positioned and “hooked-up” to the dialysis machine 40 for undergoing continuous renal replacement therapy (CRRT). The dialysis machine 40 shown is a machine designed to treat chronic renal disease and is provided with a water feed inlet 41, dialysis product line 43 and drain line 42. The patient blood line 46 passes through optoacoustic monitor 44 wherein it is monitored and analyzed as previously described. The blood is then directed into the dialysis machine via blood line 47, treated, and returned to the patient via return blood tubing line 48.

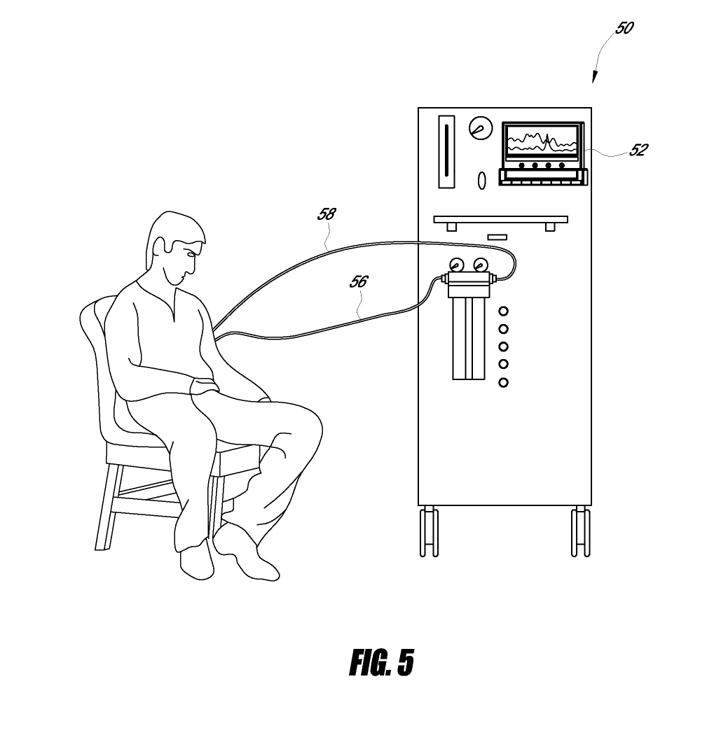

FIG. 5 illustrates a dialysis machine 50 in which the optoacoustic monitoring apparatus 52 is integrated into the dialysis machine. In this embodiment, patient blood access and return lines 56 and 58, respectively, are connected directly to the dialysis machine. In both embodiments shown in FIGS. 4 and 5, the optoacoustic monitoring apparatus comprises sensor components including the sensor module with lasers, transducers, transparent blood flow tube through which the patient's blood flows as well as the monitor, computer(s), controller(s), and other hardware and software components as previously described.

FIG. 6 illustrates an embodiment of an optoacoustic sensor monitor 62 installed along blood supply tubing in a CRRT dialysis machine tubing circuit. In the view shown, sensor module 62 is positioned along blood access tubing line 66 upstream from hemofilter 65. Also shown are effluent line 64, dialysate line 67 and blood return line 68. This tubing configuration is also disclosed in U.S. patent application Ser. No. 12/577,513, Publication No. 2010/0089806, and U.S. patent application Ser. No. 12/608,806, Publication No. 2010/0121246, the descriptions of which are incorporated herein in their entireties, respectively. In such a configuration, the monitor screen, and desired hardware and software components, other than the optoacoustic sensor module, may be conveniently positioned outside of the machine for user/operator input, control and observation.

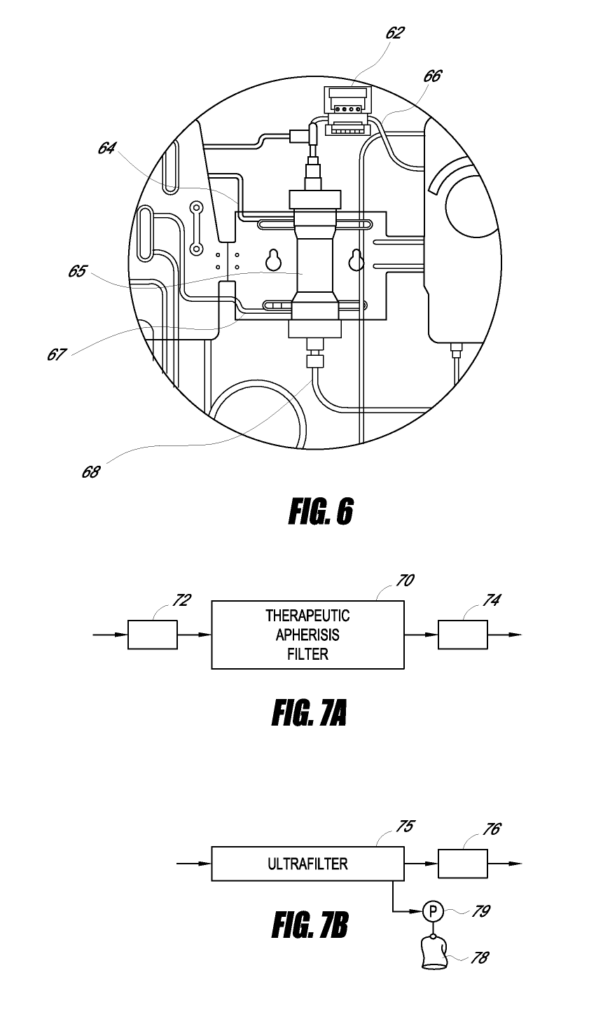

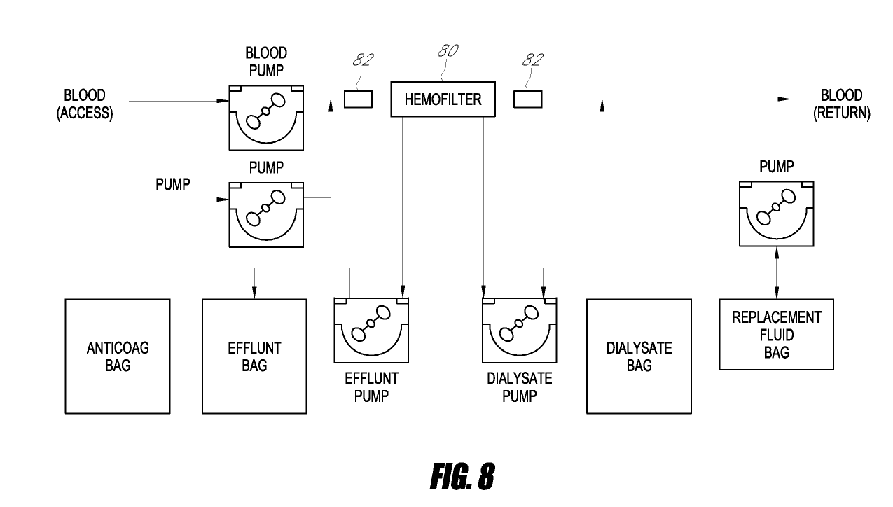

FIG. 7A schematically illustrates the use of optoacoustic detection and monitoring apparatus in a therapeutic apheresis system. As previously described, the aforesaid optoacoustic apparatus may be used in detecting and monitoring disease-related blood components, such as poisons, drugs or other harmful substances during therapeutic apheresis. For example, where patient poisoning is to be treated by such apheresis, once the poison is identified, a hemofilter 70 specific for removal of the poison may be selected and installed in the apheresis equipment. The patient's blood is then directed along the system blood flow circuit through the poison removal hemofilter. In the partial circuit illustrated in FIG. 7A, two optoacoustic monitoring apparatus 72, 74 are shown, installed upstream and downstream, respectively, of the poison removing hemofilter 70. By comparing the monitored concentrations of poison, efficiency of the poison removal therapy may be continuously monitored in real time. However, a single optoacoustic monitor may be used instead. The aforesaid poison removal is by way of example only, and such a system may be used for any desired or selected apheresis using a hemofilter configured for removal of a specific blood constituent together with one or more optoacoustic monitors. In addition to poison and drug removal, examples of diseases for which such apheresis equipment may also be used to remove pathogenic blood constituents such as listed in Exhibit 1 of U.S. Pat. No. 6,849,183 and Therapeutic Apheresis, Vol. 1, No. 2, 1997.

FIG. 7B schematically illustrates a portion of an apparatus circuit for patient fluid management. In such a system, an ultrafilter 75 configured for removing excess fluid from a patient suffering from renal failure. The treatment is referred to as slow continuous ultrafiltration (SCUF) during which the patient's blood is directed through an ultrafilter which separates and removes plasma water from the blood. In the portion of such a SCUF circuit shown in FIG. 7B, patient's blood is directed through ultrafilter 75 and separated plasma water is slowly drained to a fluid collection bag 78 via pump 79.

In the apparatus shown in FIG. 7B, an optoacoustic monitoring apparatus 76, as previously described, is installed downstream of ultrafilter 75. The apparatus may be configured to monitor the concentration of an electrolyte such as sodium or potassium in the blood stream being returned to the patient after fluid removal. The optoacoustic monitor apparatus may be configured to calculate the rate of fluid removal based on monitored sodium concentration drop or fluctuation, or determining the loss of other electrolytes, e.g., glucose, amino acids, urea, sodium chloride, from the blood stream. Moreover, a second optoacoustic monitoring apparatus may be used upstream, as shown in FIG. 7A, and comparison of one or more constituent concentrations before and after ultrafiltration may be useful in monitoring and adjusting fluid removal rates without the need for measuring the volume or the weight of removed patient fluid as is commonly practiced.

FIG. 8 schematically illustrates an apparatus configured for carrying out continuous renal replacement therapy (CRRT). Such apparatus is further described in U.S. Patent Application Publication No. 2009/0084717, incorporated herein by reference in its entirety. The dialysis apparatus schematically shown comprises an apparatus configured for performing continuous veno-venus hemodiafiltration (CVVHDF). However, other similar dialysis apparatus including systems illustrated in the aforesaid application publication for carrying out continuous ultrafiltration (SCUF), continuous veno-venus hemofiltration (CVVH) and continuous veno-venus hemodialysis (CVVHD) may also be provided with the optoacoustic monitoring apparatus and methods as previously described. Referring to the system and circuit illustrated in FIGS. 1-3, and previously described, the optoacoustic methods for detecting and determining the concentration of various important blood constituents may be used in any medical apparatus having extracorporeal access to a patient's blood flow. The optoacoustic monitoring apparatus may be installed at any one or more desired positions in the circuit. During dialysis or other blood filtration, blood may be monitored prior to and/or after filtration along the therapy circuit. In the illustration of FIG. 8, a first optoacoustic monitoring apparatus 82 is shown along the circuit upstream from the hemofilter 80, and a second optoacoustic monitoring apparatus 84 is provided downstream from the hemofilter, whereby the condition of the blood prior to filtration and after filtration may be compared, thereby giving the physician or operator an analysis of the efficiency of the hemofiltration or dialysis. Alternatively, only one such optoacoustic monitoring apparatus may be used.

The optoacoustic monitoring apparatus schematically illustrated in FIGS. 7A, 7B and 8 may be integrated with the dialysis machines and comprise optoacoustic sensor modules 32 such as described and shown in FIGS. 2 and 3 mounted along blood lines in the respective dialysis machines as shown in FIG. 6 cooperating with a monitor located conveniently elsewhere for observation. Alternatively, the optoacoustic apparatus may comprise stand-alone devices installed along blood lines upstream and/or downstream of a machine as illustrated in FIG. 4.

As previously described, the concentration of blood electrolyte constituents such as sodium, sodium chloride, potassium, phosphates, urea, glucose and/or albumin as well as combinations of two or more of such components may be of particular interest during treatment or in preventative care to determine or confirm a patient's health. The presence and concentrations of other blood constituents of interest which may be monitored include, by way of example, uric acid, B2 microglobulin and vitamin B12. A more complete list of blood electrolytes as well as other components is disclosed in U.S. Pat. No. 7,481,936, Table 1, incorporated herein by reference. It may also be useful to inject a detectable biologic marker, ligand or fluorescent dye into the patient's bloodstream upstream of the optoacoustic monitoring equipment. Such a marker or dye is configured to cooperate with the one or more of the constituents for enhancing an optoacoustic response to the laser light pulses. An example of such a dye is indocyanine green (ICG) dye, a chromophere that would be useful in optoacoustic analysis. Any detectable ligand (with or without a chromophore) may be used instead of a “dye” per se. For example, the variable section of monoclonal antibodies could be produced which bind specifically to alpha-microglobulin (or any one of a number of other blood constituents of interest). When introduced into the body, these ligands will bind only to the alpha-microglobulin, and this complex may be detected by altered acoustic responses and/or altered laser responses. Such ligands can also be made with chromophores such as fluorescein, rhodamine, or ICG adding specificity to the technique.

The optoacoustic constituent detecting and monitoring apparatus and methods may also incorporate methods and apparatus for detecting and measuring laser beam amplitude change and/or laser beam wavelength alteration or shift for one or more selected laser beams passing through a constituent containing blood flow, and correlating the amplitude change and/or wavelength alteration with corresponding constituent concentrations, respectively. Such methods are useful along with or as an alternative to the optoacoustic detectable responses in which the amplitude of acoustic waves from the one or more different ones of the constituents are measured. Where the laser beam amplitude change and/or laser beam wave length alteration or shift is monitored, the optoacoustic apparatus is supplied with appropriate sensors configured for measuring the laser beam amplitude and/or wavelength changes.

What is claimed is:

1. A method of optoacoustically monitoring blood concentrations of one or more constituents therein, comprising:
directing a flow of a patient's blood through a substantially transparent vessel, said blood having an optoacoustically detectable concentration of one or more of said constituents therein;
directing pulses of laser light into said vessel through said blood flow at one or more frequencies capable of inducing detectable acoustic responses from one or more of said constituents, respectively; and
optoacoustically detecting and measuring the induced optoacoustic response for one or more different said one or more constituents.

2. A method of claim 1, further comprising determining laser beam wavelengths that will generate optoacoustically detectable responses for selected different constituents, respectively, based on the respective constituent concentration, and determining concentrations of one or more of said blood constituents based on the respective measured optoacoustic wave frequency, slope and/or amplitudes.

3. A method of claim 2, further comprising detecting and measuring laser beam amplitude change and/or laser beam wavelength alteration for one or more selected laser beams passing through the constituent containing blood flow, and correlating amplitude change and/or wavelength alteration with corresponding constituent concentrations, respectively.

4. A method of claim 2, further comprising providing one or more pressure transducers cooperating with said transparent vessel for detecting optoacoustic responses induced from said one or more constituents in response to said laser beams.

5. A method of claim 2, further comprising providing one or more optical detectors cooperating with said transparent vessel for detecting optoacoustic responses induced from said one or more constituents in response to said laser beams.

6. A method of claim 3, further comprising providing one or more sensors configured for measuring laser beam amplitude and/or wavelength alteration.

7. A method of claim 1, further comprising determining optimum laser beam wavelengths for generating acoustic responses for one or more of said constituents, respectively, directing said optimum laser beam wavelengths into said blood flow containing vessel and detecting and measuring the optoacoustic wave frequency, slope and/or amplitude generated by said one or more constituents in response to said optimum laser beam wavelengths.

8. A method of claim 1, further comprising injecting a dye or detectable biologic marker into said patient's blood upstream of said transparent vessel, said dye or marker configured to cooperate with one or more of said constituents for enhancing an optoacoustic response to said laser light pulses.

9. A method of claim 1, wherein said constituents comprise sodium, sodium chloride, potassium, phosphate, urea, glucose, amino acids and/or combinations of two or more thereof.

10. A method of 1, wherein said constituents comprise poisons, drugs or disease-related components.

11. A method of optoacoustic monitoring blood concentrations of one or more constituents therein comprising:
directing a flow of a patient's blood through a substantially transparent vessel, said blood having an optoacoustically detectable concentration of one or more of said constituents therein;
passing pulses of laser light through said blood flow containing vessel at one or more frequencies and inducing altered laser emissions from the exposed blood and/or detectable optoacoustic waves for one or more of said constituents; and
detecting and measuring the alteration of the laser emissions and/or the frequency, slope and/or amplitude of said acoustic waves for one or more different ones of said one or more constituents.

12. A method of claim 11, further comprising determining laser beam wavelengths that will generate optoacoustic detectable responses for selected different constituents, respectively, based on the respective constituent concentration, and determining concentrations of one or more of said blood constituents based on the respective measured optoacoustic wave frequency, slope and/or amplitudes.

13. A method of claim 12, further comprising detecting and measuring laser beam amplitude change and/or laser beam wavelength alteration for one or more selected laser beams passing through the constituent containing blood flow, and correlating frequency, slope and/or amplitude change alteration with corresponding constituent concentrations, respectively.

14. A method of claim 11, further comprising determining optimum laser beam wavelengths for generating optoacoustic responses and/or altered laser emissions for one or more of said constituents, respectively, directing said optimum laser beam wavelengths into said tube and detecting and measuring optoacoustic wave amplitude and/or altered laser emissions generated by said one or more constituents in response to said optimum laser beam wavelengths.

15. A method of claim 11, further comprising injecting a dye or detectable biologic marker into said patient's blood upstream of said transparent tube, said dye or marker configured to cooperate with one or more of said constituents for enhancing the optoacoustic response to said laser light pulses.

16. A method of claim 11 wherein said constituents comprise sodium, sodium chloride, potassium, phosphate, urea, glucose and/or combinations of two or more thereof.

17. A method of claim 11, wherein said constituents comprise poisons, drugs or disease-related components.

18. A method of claim 11, further comprising providing one or more optical and/or acoustic sensors cooperating with said transparent vessel configured to detect and measure acoustic waves generated from said one or more constituents, and providing one or more sensors configured to detect and measure altered laser emissions.

19. An apparatus for monitoring concentrations of one or more constituents in a bloodstream, comprising:
means for directing a continuous flow of blood to a detection apparatus;
a detection apparatus comprising a substantially transparent vessel for receiving said continuous flow of blood, a laser light source configured for directing pulsed light into said tube which produces an optoacoustic wave response and/or measurable altered laser light emissions from said one or more constituents to said light; and
means for measuring said optoacoustic and/or said altered light response.

20. The apparatus of claim 19, wherein said means for directing comprises blood flow tubing connected to said transparent tube, and one or more pumps configured to pump blood along said blood flow tubing and through said substantially transparent vessel.

21. The apparatus of claim 19, wherein said laser light source is tunable for generating different selected wavelengths of said laser light.

22. The apparatus of claim 19, wherein said laser light source comprises a plurality of laser diodes, each said diode configured to generate a different optoacoustic response for determining concentrations of different ones of said constituents.

23. The apparatus of claim 19, further comprising one or more ultrasound detectors cooperating with said transparent vessel configured to measure the frequency, slope and/or amplitude of said optoacoustic response.

24. The apparatus of claim 23, wherein the ultrasound detector comprises a pressure transducer.

25. The apparatus of claim 23, wherein the ultrasound detector comprises an optical detector.

26. The apparatus of claim 23, further comprising software configured for measuring and/or calculating the concentrations of said one or more constituents based on the frequency, slope and/or amplitude of said optoacoustic response, respectively.

27. The apparatus of claim 19, further comprising one or more light sensors cooperating with said transparent vessel configured to measure altered laser light amplitude and/or altered laser light wavelengths.

28. The apparatus of claim 27, further comprising software configured for measuring and/or calculating the concentrations of said one or more constituents based on the laser light amplitude alteration or laser light wavelength alteration.

29. The apparatus of claim 26, further comprising one or more light sensors cooperating with said transparent vessel configured to measure altered laser light amplitude and/or altered laser light wavelengths.

30. The apparatus of claim 29, further comprising software configured for correlating altered light amplitude change and/or wavelength alteration with corresponding constituent concentrations, respectively.

31. The apparatus of claim 19, wherein said means for directing a flow of blood comprises a dialysis machine.

32. Apparatus of claim 31, wherein said dialysis machine includes a blood filter for carrying out CRRT secured along tubing for directing blood to and from a patient, and wherein said detection apparatus is positioned along said tubing upstream of said blood filter.

33. The apparatus of claim 31, wherein said dialysis machine includes a blood filter for carrying out CRRT secured along tubing for directing blood to and from a patient, and wherein said detection apparatus is positioned along said tubing downstream of said blood filter.

34. The apparatus of claim 31, wherein said dialysis machine includes a blood filter for carrying out CRRT secured along tubing for directing blood to and from a patient, and wherein said detection apparatus is positioned along said tubing upstream and downstream of said blood filter.

35. The apparatus of claim 19, wherein said apparatus comprises medical apparatus having extracorporeal access to a patient's blood flow.

36. The apparatus of claim 23, wherein said optoacoustic detector comprises a high-bandwidth ultrasound detector.

37. The apparatus of claim 36, wherein said high-bandwidth ultrasound detector comprises a piezoelectric transducer.

38. The apparatus of claim 36, wherein said high-bandwidth ultrasound detector comprises an optical etalon.

39. The apparatus of claim 36, wherein said high-bandwidth ultrasound detector comprises a Fabry-Perot interferometer.

40. The apparatus of claim 19, further comprising means cooperating with said optoacoustic detection apparatus for processing and transmitting said optoacoustic response to a remote location.

41. The apparatus of claim 40, further comprising means for displaying said optoacoustic response at said remote location.

42. The apparatus of claim 40, wherein said means comprises a microprocessor or computer cooperating with a modem for processing, recording and transmitting optoacoustic response data to a remote location.

43. The apparatus of claim 41, wherein said means for displaying comprises a monitor and/or printer at said remote location for displaying the optoacoustic response data.
